(19) United States
(12) Patent Application Publication
(10) Pub. No.: US 2007/0134665 A1
(43) Pub. Date: Jun. 14, 2007

(54) Method of in situ detection of proteins using aptamers

(75) Inventors: Graham Dellaire, Toronto (CA); David P. Bazett-Jones, Toronto (CA)

Correspondence Address: SIM & MCBURNEY, 330 UNIVERSITY AVENUE, 6TH FLOOR, TORONTO, ON, M5G 1R7, CA

(73) Assignee: THE HOSPITAL FOR SICK CHILDREN, Toronto, ON (CA)

(21) Appl. No.: 10/574,018

(22) PCT Filed: Sep. 29, 2004
(86) PCT No.: PCT/CA04/01757

§ 371 Date: Dec. 1, 2006

Related U.S. Application Data
(60) Provisional application No. 60/506,776, filed on Sep. 30, 2003.

(51) Int. Cl. C12Q 1/70; C12Q 1/68

(52) U.S. Cl. 435/6; 435/5; 977/924

(57) ABSTRACT

The present invention is a method for the detection and localization of proteins in situ. The method comprises contacting a peptide tag labelled protein with a nucleic acid aptamer that recognizes the peptide tag. The nucleic acid aptamer is functionalized such that is detectable by a variety of methods. The method allows for the detection of one or more proteins and/or protein complexes in sub-cellular and sub-nuclear structures with high specificity and also allows for the purification of such identified proteins.

### FIELD OF THE INVENTION

The invention relates to the detection and localization of proteins. More specifically, the present invention is directed to a method for the in situ detection and localization of one or more proteins or protein complexes with specificity within cellular sub-structures at high resolution. The method is herein referred to as Aptamer In Situ Detection, (Aptamer-ID™) or Aptamer protein isolation (Aptamer-PI™).

### BACKGROUND OF THE INVENTION

Cellular proteins are often detected using conventional antibody techniques such as with the use of antibodies directed against the proteins themselves or to peptide tags fused to these proteins (e.g. Flag-tag or HA-tag, Terpe, 2003). The antibodies can be functionalised with fluorescent molecules or gold particles in order that the protein of interest be recognised by light (LM) and electron microscopy (EM). To visualise the localisation and dynamics of specific proteins in living cells, natural and engineered fluorescent proteins (FPs) (e.g. green fluorescent protein, “GFP”) have been used as tags (Pines, 1995; van Roessel and Brand, 2002). While both antibodies and FPs have been attractive reagents for cell biologists because of their versatility and ease of use, several limitations with these systems exist. One limitation is the small repertoire of bright and spectrally distinct proteins or fluorophores that are available. In addition, a specific combination of excitation filter, emission filter and dichroic mirror is required to visualise the fluorescence emission from a particular fluorophore or combination of fluorphores during epifluorescence microscopy, with the result that detection of three different proteins is the practical limit. Furthermore, the detection of three or more proteins simultaneously in situ using antibodies requires that each antibody be produced in a different and immunologically distinct animal so that secondary antibodies conjugated to each fluorophore do not cross-hybridise, resulting in false positive detection.

Prokaryotic DNA binding proteins have been previously used to image in situ nuclear structures such as chromatin. For example, Robinett et al., (1996) discloses the use of the LacI repressor to detect chromatin sequence in vivo carrying a large array of Lac operator DNA sequences. Michaelis et al., (1997) discloses a similar method of imaging chromatin sequences in vivo using the TetR repressor to detect an array of Tet operator DNA sequences located in a yeast chromosome. These methods use protein to detect nucleic acid sequences in chromatin. These methods require the manufacture of multiple constructs and multiple labelling which is difficult, thus in situ hybridization is stated to be required for allowing comparisons with native chromosome structure. These methods are only applicable to the imaging of chromatin and thus do not represent a general imaging technology for proteins.

Aptamer technology has developed over the past decade. This technology relies on the use of single or double stranded DNA or RNA oligonucleotides called aptamers, which can bind protein targets with high affinity (Brody and Gold, 2000). Aptamers that recognise a specific protein are selected by multiple rounds of binding, isolation and amplification using a procedure termed systematic evolution of ligands by exponential enrichment (SELEX™) (Tuerk and Gold, 1990; Ellington and Szostak, 1990, U.S. Pat. No. 5,270,163 and U.S. Pat. No. 5,475,096).

Aptamers can be functionalised and used much in the same way as antibodies, for example in ELISAs, sandwich assays and Western blotting (Bacher and Ellington, 1998; Jayasena, 1999; Morris et al., 1998). Aptamers have been selected that bind a number of eukaryotic transcription factors (Roulet et al., 2002) as well as non-DNA binding proteins such as thrombin (Bock et al., 1992 and Dougan et al., 2003). In general, aptamers are much smaller than F′ab fragments of antibodies (<10 kDa on average compared to 50 kDa, respectively, Stanlis and McIntosh, 2003).

Various references, patent applications and patents have described different methods employing aptamers for the detection of various materials. For example, Bianchini et al., (2001) discloses the use of aptamers to directly recognize native and denatured proteins. Blank et al., (2001) discloses the use of a DNA aptamer to bind to rat brain tumor microvessels.

Stanlis and McIntosh (2003) describes an attempt of the use of single-strand DNA aptamers bound to a green fluorescent protein (GFP) as probes for protein localization in cells. However, the proposed method proved unsuccessful.

U.S. Pat. No. 6,261,783 discloses a method for detecting target molecules in mixtures using a double stranded aptamer capable of binding to the target molecule and wherein once bound to the target molecule, the aptamer unwinds such that each of its two strands may become available for extramolecular hybridization. The mixture is then contacted with first, second and third cascade nucleic acids so that the nucleic acid strands of the aptamer triggers a cascade of intermolecular hybridization leading to formation of a multimolecular hybridization complex which is then detected.

U.S. Pat. No. 6,287,765 discloses a method for detecting oligonucleotide molecules that bind to non-oligonucleotide molecules. The method comprises contacting a selected non-oligonucleotide molecule with a mixture of non-naturally occurring oligonucleotides and detecting a non-naturally occurring oligonucleotide molecule that is bound the selected non-oligonucleotide molecule by a method that may be selected from scanning probe microscopy, optical trapping and flow cytometry.

U.S. Pat. No. 6,531,286 and U.S. patent application 2001/0055773 disclose a ligand beacon assay involving the interaction of an aptamer (nucleic acid ligand) with a molecular beacon (ligand beacon) whose nucleotide sequence in the loop is complementary to a nucleotide stretch in the aptamer. This interaction causes the spatial separation of the fluorophore (star) from the quencher (pentagon) producing a fluorescence signal.

While the aforementioned prior art disclose various methods of using DNA aptamers to detect proteins and other molecules, it is always desirable to provide new improved methods that further improve the efficiency of protein detection, provide the capacity for multiplex detection and provide for the detection of proteins in situ within sub-cellular structures at high resolution and thus overcoming disadvantages of using prior art methods.

### SUMMARY OF THE INVENTION

The present invention is a method of detecting proteins in situ wherein a peptide tag is used that can bind a double stranded nucleic acid aptamer with high affinity. The peptide tag is expressed as a fusion protein encoding both the tag and a known protein, which protein is desired to detect/localize in situ. The peptide tag portion of the fusion protein is recognized/binds to its cognate nucleic acid aptamer which itself is functionalized so that it is readily detectable by a variety of methods. The nucleic acid aptamer sequence thus detects proteins in situ which has not previously been contemplated. The method of the invention provides for the detection and localization of a protein in situ with specificity within sub-cellular and sub-nuclear structures at high resolution. The method also allows for the multiplex visualization of sub-cellular and sub-nuclear complexes of proteins within a cell. The method can also be used for protein purification for both proteomic and therapeutic applications. Further, the method allows for detection of different proteins and protein complexes in the same cell without cross-hybridization of the detecting nucleic acid aptamers. Lastly, only nanomolar quantities of DNA aptamer are required in the present method. Detected proteins can then be further isolated and purified.

In accordance with an aspect of the present invention is a method for detecting a protein in situ, the method comprising contacting a peptide tag fusion protein with a nucleic acid aptamer that recognizes the peptide tag. The nucleic acid aptamer is functionalized such that is detectable by a variety of methods.

According to another aspect of the present invention is a method for the detection of one or more proteins and/or protein complexes in situ, the method comprising contacting a nucleic acid aptamer with a fusion protein comprising a protein tag, wherein said protein tag binds to the nucleic acid aptamer to form a complex, and detecting the complex.

According to yet a further aspect of the present invention is a method for the detection of one or more proteins and/or protein complexes in situ, the method comprising contacting a functionalized nucleic acid aptamer with a fusion protein comprising a protein tag, wherein said protein tag is recognized by the functionalized nucleic acid aptamer and forms a complex, and detecting said complex.

According to another aspect of the present invention is a method for the detection of one or more proteins and/or protein complexes in situ, the method comprising contacting a nucleic acid aptamer with a fusion protein comprising a protein tag, wherein said protein tag is recognized by the nucleic acid aptamer and wherein said protein tag is a prokaryotic DNA binding protein. The nucleic acid aptamer may be functionalized such that it is detectable by a variety of methods.

According to yet another aspect of the invention is a method for the detection of one or more proteins and/or protein complexes in situ, the method comprising:
contacting a transfected cell expressing a fusion protein comprising a prokaryotic DNA binding protein with a functionalized nucleic acid aptamer, wherein said protein tag is recognized by said functionalized nucleic acid aptamer to form a complex, and detecting said complex.

According to still another aspect of the present invention is a method for detecting proteins and/or protein complexes in situ, the method comprising;

a) preparing a protein/protein tag fusion vector;

b) transforming a mammalian cell with a);

c) contacting b) with a functionalized nucleic acid aptamer, wherein said protein tag is recognized by said nucleic acid aptamer and forms a complex; and

d) detecting said complex.

According to yet another aspect of the present invention is a method for purification of protein or protein complex in vitro, the method comprising;

a) transforming a mammalian cell with a protein/protein tag fusion vector;

b) contacting (a) with a functionalized nucleic acid aptamer wherein said protein tag is recognized by said nucleic acid aptamer and forms a complex;

c) preparing a cell lysate of (b); and

c) detecting said complex.

In aspects, the nucleic acid aptamer is functionalized with biotin and detected with the use of streptavidin beads.

According to still another aspect of the invention is a kit for the detection and localization of a protein in situ, said kit comprising:
one or more vectors comprising a peptide tag and cDNA sequence encoding a desired protein, wherein said one or more vectors is expressed when transformed in a cell to provide a fusion protein comprising the peptide tag and desired protein; and
one or more aptamers specific for binding to said peptide tag.

The kit may further include instructions for use. In further aspects, each vector may contain more than one peptide tag and cDNA protein coding sequence.

According to still another aspect of the invention is a kit for the purification of a protein, said kit comprising:
a vector comprising a peptide tag and cDNA sequence encoding a desired protein, wherein said vector is expressed when transformed in a cell to provide a fusion protein comprising the peptide tag and desired protein; and
a labelled aptamer specific for binding to said peptide tag, said labelled aptamer being covalently coupled to an affinity matrix or beads.

In this aspect, a streptavidin/biotin interaction can be used as the detection system.

In other aspect of a protein purification kit, the aptamer is coupled to paramagnetic particles facilitating purification of the fusion protein using a magnet.

The kit may further include instructions for use.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from the detailed description.

### BRIEF DESCRIPTION OF THE DRAWINGS

The present invention will become more fully understood from the detailed description given herein and from the accompanying drawings and figures, which are given by way of illustration only and do not limit the intended scope of the invention.

FIG. 1 shows an overview of Aptamer-ID and Aptamer-PI for in situ detection and purification of proteins. FIG. 1A) Aptamer-ID of proteins in situ. Cells grown on slides are fixed and hybridised with fluorescently labelled DNA aptamers (red or green) that can detect the presence of proteins fused to either LacI (1) or TetR (2). The fluorescent aptamers corresponding to the operator sequences for LacI or TetR (green or red, respectively) bind each bacterial fusion protein specifically without cross hybridising resulting in the sub-nuclear localisation of the tagged proteins by light microscopy. FIG. 1B) Aptamer-PI for the detection of proteins in vitro. Streptavidin-sepharose beads (S) are sequentially incubated with biotinylated DNA operators specific for LacI followed by incubation with a cell lysate containing LacI fused to a protein of interest (Terpe, K. 2003, Appl. Microbiol. Biotechnol., 60, 523-533). After binding the beads are washed and the resulting purified LacI-fusion protein may be observed by SDS-PAGE. M=marker, IN=input lysate, PI=isolated protein.

FIG. 2 shows the design of LacI-fusion vectors for Aptamer-ID™. FIG. 1(A) pGD-Flag-Lac338 contains the first 338 amino acids of LacI (LacI-338), with a single N-terminal Flag-tag epitope. The protein sequence of interest is cloned downstream of the KpnI site and expression of the resulting LacI-fusion proteins is driven by a cytomegalovirus promoter (cmv). FIG. 2(B) pGD-Flag-Lac338-SC35 contains the full length human SC35 gene (Hs.SC35) fused in the same reading frame as the LacI repressor. FIG. 2(C) pGD-HA-TET contains the full length TetR gene, with a single N-terminal HA-tag epitope. The protein sequence of interest is cloned downstream of the BamHI site and expression of the resulting TetR-fusion proteins is driven by a cytomegalovirus promoter (cmv). FIG. 2(D) pGD-HA-TET-PML contains the full length human PML IV gene (Hs.PML IV) fused in the same reading frame as the TetR repressor.

FIG. 3 shows In situ localisation of LacI-tagged SC35 in human SK-N-SH cells by Aptamer-ID™. FIG. 3(A) Localisation of the LacI-tagged SC35 in paraformaldehyde fixed cells transfected with pGD-Flag-Lac-SC35 using the anti-Flag antibody M2 (Flag, red) or FIG. 3(B) with a Cy3-labelled dsDNA aptamer (O-Sym, red) specific for LacI. PRP4 kinase was also localised as an endogenous marker for splicing speckles using-a sheep polyclonal antibody (PRP4K, green). Positive co-localisation between PRP4K and LacI-tagged SC35 is demonstrated by a yellow signal in the merged images. The localisation of the LacI-SC35 fusion protein was observed only in transfected cells (FIG. 3B, O-Sym) and showed complete co-localisation with PRP4 kinase (PRP4K, green) in nuclear speckles. DNA was counterstained with DAPI (blue in merged images). Scale bars represent 5 μm

FIG. 4 shows In situ localisation of TetR-tagged PML and LacI-tagged SC35 in human SK-N-SH cells by Aptamer-ID™. FIG. 4(A) shows the localisation of the TetR-tagged PML in paraformaldehyde fixed cells transfected with pGD-HA-TET-PML using the anti-HA (HA, red) or FIG. 4(B) with a Cy5-labelled dsDNA aptamer (TET-O, red) specific for TetR. Endogenous PML was also localised as a marker PML nuclear bodies using a rabbit polyclonal antibody (PML, green). Positive co-localisation between PML and TetR-tagged PML is demonstrated by a yellow signal in the merged images. The localisation of the TetR-PML fusion protein was observed only in transfected cells (see B, TET-O) and showed complete co-localisation with PML (PML, green) in PML nuclear bodies. FIG. 4(C) Multiple detection and localisation LacI-SC35 and TetR-PML in cells transfected with pGD-HA-TET-PML and pGD-Flag-Lac338-Sc35. Localisation LacI-SC35 and TetR-PML was accomplished using Cy3-labelled O-Sym or Cy5-labelled TET-O dsDNA aptamers, respectively. TetR-PML and LacI-SC35 do not co-localise (separate red and green signals (respectively) in merged image), thus demonstrating the utility of Aptamer ID™ for multi-plex detection of proteins in situ. DNA was counterstained with DAPI (blue in merged images). Scale bars represent 5 μm

FIG. 5 shows Aptamer-PI of LacI-tagged SC35 from human SK-N-SH cells. Total cellular lysates and isolated protein (PI) lysates were prepared from SK-N-SH cells transiently expressing LacI or LacI-tagged SC35. The protein isolation of LacI and LacI-tagged SC35 from PI-lysates was carried out using streptavidin beads pre-incubated with biotinylated dsDNA aptamer specific for LacI (O-Sym). Western analysis of total cellular lysate (1), PI lysate (2), Aptamer-PI (3), and mock PI (4) from LacI-SC35 expressing cells, or Aptamer-PI from PI lysate generated from LacI expressing cells. Proteins were detected using either anti-Flag (A) or anti-SR protein (mAb 104) (B) antibodies. The black arrow indicates the position of LacI-SC35 in panel B. In addition, a Coomassie stained SDS-PAGE gel is shown as a qualitative comparison of Aptamer-PI versus immunopreciptiation (IP) using an anti-Flag antibody (C). In panel C: lane M=protein molecular weight marker; lane 1=40 μg of total lysate; lane 2=Aptamer-PI; lane 3=Mock PI; and lane 4=anti-Flag IP. An asterix marks the position of co-purifying immunoglobulin (i.e. heavy chain is shown) in the Flag-IP. The black arrows indicate the position of LacI-SC35 in panels B and C.

FIG. 6 shows correlative light and electron spectroscopic imaging (LM/ESI) of SK-N-SH cells expressing LacI-tagged SC35 using fluorogold Aptamer-ID. Detection of nuclear speckles (arrow) via the Cy3 fluorophore (FIG. 6A, left panel), and confirmation of gold conjugation to the aptamer through silver enhancement (FIG. 6A, right panel). Untransfected cells (arrowhead) contain background levels of silver deposition. Scale bar=200 nm. FIG. 6B) Overlay of fluorescence and low magnification ESI micrographs collected at 155 eV. With two rounds of the silver enhancement using a LM enhancement kit (FIG. 6B, left panel), the silver particles are visible as bright spots. The right panel is an overlay image of a nucleus containing EM-enhanced gold. Scale bar=2 μm. An area corresponding to an IGC, as defined by a box in the left panel is analysed at high resolution, and maps of phosphorus (FIG. 6C, red panel) and nitrogen (FIG. 6C, green panel) show the ultrastructure of the IGC region, which is low in phosphorus content and contains protein-based fibrous structures. The fluorogold aptamer localisation is indicated with silver-enhanced gold particles false-coloured in white. The composite map (FIG. 6D, left panel) illustrates the position of the IGC relative to chromatin (Ch, yellow) and the nucleolus (Nu, yellow-green). The silver particles in the interior of the IGC are labelled with arrowheads, whereas those proximal to the neighbouring chromatin are indicated by arrows. The composite map (FIG. 6D, right panel) of the EM-enhanced nucleus (FIG. 6B, right panel) contains smaller but uniform silver particles, as indicated by the arrows and arrowheads. Scale bars=0.5 μm.

### DETAILED DESCRIPTION OF THE PREFERRED EMBODIMENTS

The Applicant has developed a new method of detecting proteins in situ, herein referred to as Aptamer in situ detection (Aptamer ID™). The method is based on the transfection of a suitable cell with a protein-peptide tag fusion vector that can be recognized by a double stranded nucleic acid aptamer with high affinity. It is the peptide tag portion of the fusion protein that is recognized by the nucleic acid aptamer. The nucleic acid aptamer is functionalized such that it can be detected by a variety of methods. The method of the invention may in aspects be used to purify proteins for proteomic and therapeutic applications and in this aspect is referred to herein as Aptamer-PI™ (protein isolation),

The Aptamer-ID™/Aptamer-PI™ method of the invention is rapid and flexible, being less time consuming to complete than conventional immuno-detection methods employing primary and secondary antibodies. The present method is easily combined with the standard antibody detection protocol without extensive sample processing. Furthermore, the method of the invention provides the ability for multiplex detection of proteins in situ alone or in combination with existing protocols for immunofluorescence using antibodies. The method of the invention allows for the detection of proteins in vitro and in situ (both sub-cellular and sub-nuclear structures at high resolution) using several methods including LM and EM as well as for the isolation and purification of the tagged protein.

In the method, a protein-peptide tag fusion vector is first made and transfected into a suitable host cell in order that the fusion protein be properly expressed. In the method, any desired cDNA sequence encoding a desired protein may be employed and inserted into a suitable vector system using standard methodology as is known by one of skill in the art (Short Protocols in Molecular Biology, 4th Edition by Frederick M. Ausubel, Roger Brent, Robert E. Kingston, David D. Moore, J. G. Seidman, John A. Smith, Kevin Struhl, John Wiley & Sons, Apr. 19, 1999). The selection of the protein is only limited to size with respect to the particular vector system used as is well understood by one of skill in the art. It is also within the scope of the present invention to construct a fusion vector containing more than one protein-peptide tag such that more than one fusion protein is expressed. Alternatively, more than one protein-peptide tag fusion vector, each expressing a different fusion protein, may be transfected into a single cell type for detection with a suitable aptamer for multiple protein localization.

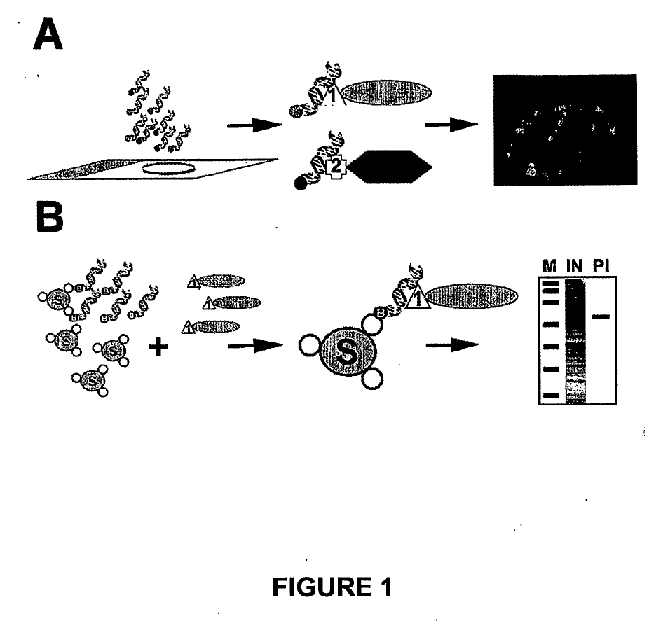

A non-limiting overview of the method of the invention is shown in FIG. 1 with respect to the use of the Lac repressor (LacI) and the Tet repressor (TetR). The method can be used with respect to detecting proteins in cultured cells (FIG. 1A) or to detect/purify proteins in vitro from cell lysates (FIG. 1B). In this non-limiting representative overview of the invention, the Lac repressor (LacI) and the Tet repressor (TetR) were chosen as aptamer-binding protein tags to demonstrate the ability of nucleic acid aptamers to image proteins in situ within cells. A LacI-fusion vector was generated (pGD-Flag-Lac338) containing the sequence of the first 338 amino acids of LacI downstream of a single Flag-tag. A second DNA construct, a TetR-fusion vector was constructed (pGD-HA-TET) containing the full length Tet repressor downstream of a single hemagglutinin (HA) epitope-tag. The cDNA sequence of any protein can then be cloned into these vectors for the expression of LacI-or TetR-tagged fusions of the target protein in mammalian cells. Consequently, the fusion protein can be localised using a fluorescently labelled dsDNA aptamer specific for the LacI (i.e. O-Sym) or TetR protein (i.e. TET-O)(see examples).

The peptide tag as used herein may be any natural or engineered DNA binding protein or an engineered or naturally derived peptide for which nucleic acid aptamers have been selected to bind with high affinity. Naturally derived peptide-tag sequences are understood to include peptides found within the amino acid sequence of any virus, archaebacteria, eukaryotic or prokaryotic organism. In one aspect, the peptide tag sequence suitable for use in the method of the invention is selected from a prokaryotic DNA binding protein. The peptide sequence may be modified by methylation, acetylation, phosphorylation, ADP-ribosylation, sumolation, ubiquitination, glycosylation, hydroxylation and any combination of these and other modifications as is understood by one of skill in the art. In one aspect, the peptide tag may be about 4 amino acids or greater in length such as to facilitate detection, localization and purification of the tagged fusion protein.

The selection of the peptide sequence for use as a peptide tag is further only limited to the extent that it should not share extensive homology to proteins within the host cell and therefore artificial or cross-species selection is preferred. In this manner, it is less likely that non-specific binding within the cell will occur if peptides are chose from evolutionary distant species to the host cell (i.e. bacterial peptides used in imaging proteins in mammalian cells). Non-specific binding may be tested empirically for each peptide sequence selected as is understood by one of skill in the art.

The Lac repressor is the prototype of a large family of prokaryotic helix-turn-helix (HTH) DNA binding proteins, the sequences of which are available for over 25 members including the fructose repressor (FruR), the purine repressor (PurR), and the galactose repressor (GalR) (Nguyen and Saler, 1995). In addition, the TetR, AraC, MerR, and MarR families of DNA binding proteins involved in multi-drug transport and resistance in prokaryotes provide additional examples of HTH-containing DNA binding proteins that bind specific operator sequences (Grkovic et al., 2002). Several of these proteins including TetR, BmrR, QacR, and EmrR have been studied extensively both biochemically and by X-ray crystallography and may be used in the method of the present invention.

Prokaryotic DNA binding proteins provide several advantages for use as peptide tags in the method of the present invention. Firstly, many prokaryotic DNA binding proteins have evolved as components of regulatory operon systems that sense the presence of small molecules and metabolites. These small molecules modulate the binding of such proteins to their DNA operator sequences through binding to regulatory domains within these transcription factors and repressors. For example, isopropyl beta-d-thiogalactoside (IPTG) can reduce the affinity of LacI for its operator in a dose dependent manner (Kercher et al., 1997). Similarly for TetR, the presence of tetracycline modulates the interaction of TetR with its operator (Hillen and Berens, 1994). Thus, it may be possible to modulate the sensitivity of in situ detection by addition of these small molecules and metabolites. Lastly, because of the evolutionary distance between prokaryotes and higher eukaryotes such as mammals, the existence of high affinity operator sequences within the mammalian genome would be unlikely, thus fusion proteins encoding these prokaryotic peptides are less likely to mislocalize by binding to genomic sequences. Taken together, these and other prokaryotic DNA binding proteins may be used as aptamer-peptide tag pairs for the present multi-plex Aptamer-ID™/Aptamer-PI™ method of the invention.

The selection or production of the aptamer for use in the method of the invention is done by methods known to those of skill in the art (Tuerk and Gold 1990; Ellington and Szostak 1990, herein incorporated by reference in their entirety). Suitable aptamers for use in the invention may be a single stranded, double stranded or hairpin DNA; single stranded, double stranded or hairpin RNA; protein nucleic acid aptamer (PNA); or any combination or hybrids of these molecules so long as the molecule can recognize and bind the peptide tag with high affinity. It is also understood by one of skill in the art that double stranded DNA or RNA includes double strands having a single stranded extension on either end. In other words, one or both of the strands may be extended at either end creating a tail portion. In aspects of the invention, the aptamer is double stranded. The nucleic acid aptamer backbone may be further modified or contain modified sugars or bases or be provided with enzymatic activity such as in the case of a ribozyme. In aspects of the invention, the aptamer is about 10 base pairs in length or longer. The nucleic acid sequence of the aptamer may be a naturally occurring nucleic acid sequence derived from a virus, archaebacteria, prokaryote or eukaryote or may be specifically selected according to the SELEX™ methodology. The SELEX process uses large (10¹⁴-10¹⁵ sequences) oligonucleotide pools to identify binding species, i.e. aptamers to a variety of purified molecular targets such as proteins/small molecules, cells and tissues. Selection against purified protein allows optimal enrichment of high-affinity aptamers (Irvine et al., 1991, J. Mol. Biol. 222, 739-761).

The nucleic acid aptamers may be chemically modified (i.e. functionalized) with a variety of moieties, including reactive thiols, amines, cobalt or iron paramagenetic beads, fluorophores, quantum dots, peptides, radio-isotopes, metal chelating peptides or compounds, simple or complex sugars and biotin using methods that are well known to those of skill in the art. Such functionalization allows for the ultra-structural analysis of nuclear or cytoplasmic structures within a cell containing the peptide-tagged protein or an endogenous protein whose amino acid sequence contains the peptide-tag sequence to which the nucleic acid aptamer binds with high affinity. In addition, multiplex detection of more than 24 aptamers simultaneously can be achieved by modifying individual aptamers with multiple fluorphores as is well known to those skilled in the art of spectral karyotyping (SKY) and multiplex fluorescent in situ hybridisation (M-FISH) (Schrock et al., 1996; Speicher et al., 1996). Furthermore, the nucleic acid aptamer can be fixed to a solid support such as to sepharose beads by covalent (e.g. thiol, amine chemical coupling or use of cross-linking reagents) or non-covalent means (i.e. paramagnetic beads, or biotin-streptavidin) to any surface to facilitate chip-based detection, column or batch purification of the peptide tagged protein or naturally occuring protein containing the peptide-tag as well as by immunoprecipitation and purification of any interacting peptides. In aspects of the invention only nanomolar amounts of nucleic acid aptamer are required in the invention, however, it is understood by one of skill in the art that various amounts of nucleic acid aptamer can be used in the method of the invention such as for example, but not limited to 1 nM to about 10 μM range and any range therebetween.

In one aspect of the invention, fluorescently modified and biotinylated oligonucleotide aptamers are used for light microscopy, however, it is possible to directly couple other moieties also useful for electron microscopy such as gold or other metal complexes. In one particular embodiment of the invention, the use of dsDNA aptamers allows the detecting molecule to be functionalised in four independent reactions during oligosythesis (i.e. at the 3′ and 5′ of each DNA strand). In addition, further functional diversity may be accomplished by the direct coupling of peptides with desirable properties to the aptamer, such as specific reactive groups or side chains (e.g. additional amine groups via poly-lysine). Thus, it is within the scope of the present invention to create pluri-functional aptamers for use with the Aptamer-ID™ technology. For example by utilising a fluorophore on one DNA strand and nanogold (i.e. gold modified) coupled to a second DNA strand, the resulting dsDNA aptamer may be used for correlative light microscopy (through the fluorophore) and electron microscopy (through post-detection silver enhancement of the gold moiety).

In the invention, a protein-peptide tag fusion vector is constructed and transfected into a suitable cell for expression of the fusion protein. Again, any mammalian cell line can be used as is understood by one of skill in the art. The cell line selected should be selected that does not endogenously express the protein tag. It is understood by one of skill in the art that any desired. cDNA coding sequence can be selected for transfection as desired and thus for detection and/or purification.

Once the cells are suitably incubated for a time period sufficient to express the fusion protein, the cells are fixed using standardized fixation methods incorporating fixatives such as ethanol, methanol or bifunctional cross-linking fixatives such as formaldehyde, paraformaldehyde and glutaraldehyde, which preserve more adequately the structures of cells. Other fixatives include water-soluble carbodiimide and the bifunctional reagent parabenzoquinone. Fixation conditions are readily conformed to the particular aptamer being used in order that the aptamer not be adversely affected and can bind to the peptide tag with high specificity.

Prior to incubation with the desired aptamer, the fixed cells are permeabilized using suitable agents (for example but not limited to Triton-X) as is well understood by one of skill in the art. After a suitable time of incubation in the presence of the functionalized aptamer, the cells are subjected to a variety of detection methods for visualization and localization of the tagged protein. The detection method used is dependent on the specific type of functionalization to which the aptamer has been subjected. A desired detection method is immunofluorescence, where the aptamer has been modified with the use of biotin or Cy3 for example.

While the invention has been described with respect to the use of fixed cells (i.e. not living), it is possible to incorporate the method of the invention to living cultured cells by techniques such as microinjection (Molecular Genetics, Ulrich Melcher© 1999), lipofection (Journal of Molecular Medicine, Vol 75, Issue 3, 1997, pgs. 223-229) electroporation or other means of introducing the detecting oligonucleotide into living cells as is understood by one of skill in the art. Living cells may then be visualized using standard microscopic methods.

In another embodiment of the invention, the aptamer peptide-tag fusion method of the invention can be used for the purification of proteins for proteomics analysis such as mass spectrometry especially where the protein complexes of interest are isolated under native conditions. This is herein referred to as Aptamer-PI (aptamer protein isolation). For example, the tandem affinity purification (TAP) tag system, which utilises tandem IgG binding domains from protein A and the calmodulin binding peptide, allows the elution of proteins under native conditions using EGTA (Puig, et al., 2001, Methods, 24, 218-229). In Aptamer-PI, which is based on TetR or LacI fusion proteins, gentle elution from the affinity column may be accomplished by the addition of heparin (Gadgil et al., 1999, J. Chromatogr. A. 848, 131-138) to the column or by using small metabolites such as tetracycline (Hillen et al., 1994, Annu Rev. Microbiol., 48, 345-369) or isopropyl beta-d-thiogalactoside (IPTG)(Gadgil et al., 2001, J. Biochem. biophys), respectively. In particular, affinity purification of proteins by nucleic acid based chromatography may provide a means of isolation of recombinant proteins for therapeutic use, as antibody-based chromatography runs the risk of the introduction of antibody fragments that may induce an undesirable antigenic response in the patient. Thus, Aptamer-PI™ may provide an ideal mode of protein purification for both proteomic and therapeutic applications.

Detection of LacI-Tagged Splicing Factor SC35 and TetR-Tagged Promyelocytic Leukaemia Protein PML

In one aspect of the invention, two well known prokaryotic DNA binding proteins, the Lac repressor (LacI) and the Tet repressor (TetR) were selected as peptide tags and the symmetric double stranded DNA (dsDNA) Lac operator (O-Sym) or the dsDNA Tet operator (Tet-O) as the detecting aptamers. The Aptamer-ID™ method was used to detect a LacI-tagged splicing factor, SC35 and a TetR-tagged promyelocytic leukaemia protein (PML). SC35 localises to very well characterised sub-domains within the mammalian nucleus, termed nuclear speckles. These structures contain both RNAs and proteins involved in pre-mRNA splicing and metabolism (reviewed in Spector 1993). The results demonstrate that LacI-tagged SC35 and TetR-tagged PML localise properly to nuclear speckles and PML nuclear bodies (respectively) and that these proteins can be detected in situ using small (41 bp) dsDNA aptamers at nanomolar concentrations. Furthermore, both of these proteins were detected in the same cell without cross-hybridisation of the detecting aptamers, demonstrating the ability of the present Aptamer-ID™ method for multiplex detection of proteins in situ. Thus, the results provide the first evidence that nucleic acid aptamers can be used to localise proteins in situ within sub-cellular structures at high resolution.

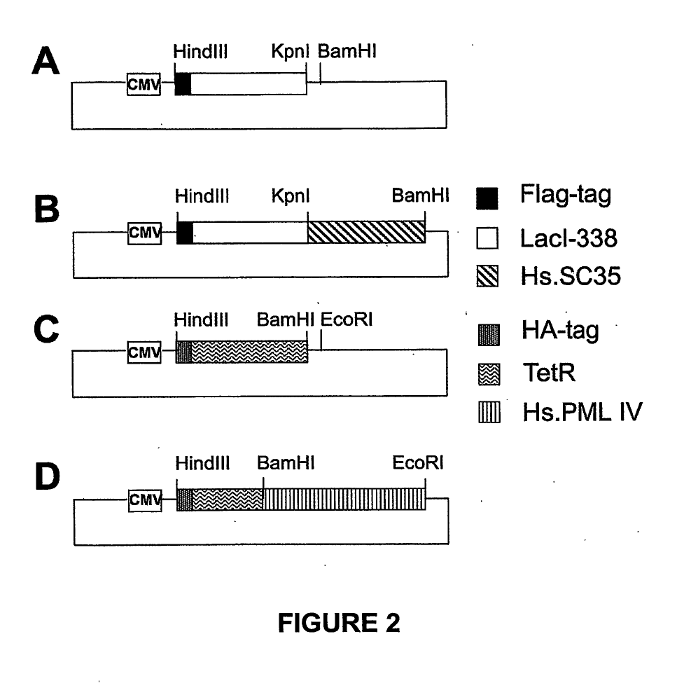

The Lac repressor (LacI) and the Tet repressor (TetR) were chosen as aptamer-binding protein tags to demonstrate the ability of nucleic acid aptamers to image proteins in situ within cells. A LacI-fusion vector was generated (pGD-Flag-Lac338) containing the sequence of the first 338 amino acid of LacI downstream of a single Flag-tag (FIG. 2A). This truncated form of the LacI protein is incapable of forming tetramers and is thought to form dimers in vivo (Robinett et al., 1996). A second DNA construct, a TetR-fusion vector was constructed (pGD-HA-TET) containing the full length Tet repressor downstream of a single hemagglutanin (HA) epitope-tag (FIG. 2C). The cDNA sequence encoding the human SC35 gene was cloned into the LacI expression vector to create pGD-Flag-Lac-SC35 (FIG. 2B). A human neuroblastoma cell line, SK-N-SH, was transfected with pGD-Flag-Lac-SC35. The cDNA sequence of any protein can then be cloned into these vectors for the expression of a LacI-or TetR-tagged fusions of the target protein in mammalian cells. Consequently, the fusion protein can be localised using a fluorescently labelled dsDNA aptamer specific for the LacI (i.e. O-Sym) or TetR protein (i.e. TET-O).

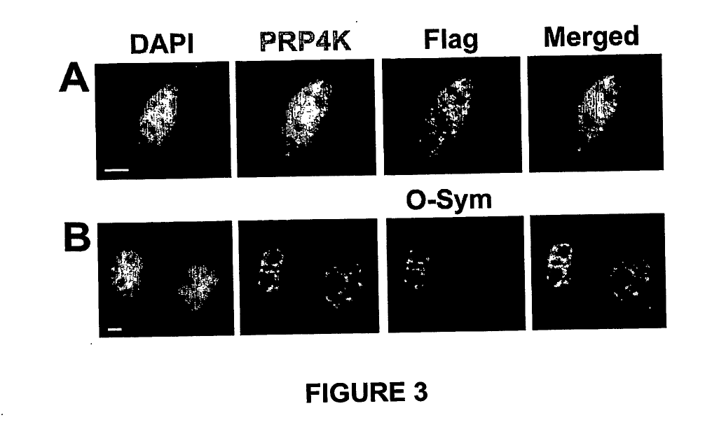

After 24 hours post transfection, cells were fixed and analysed by immunofluorescence using antibodies directed against the Flag epitope or with the O-Sym aptamer (FIG. 3). This localisation was compared to that of PRP4 kinase (PRP4K), an endogenous marker for nuclear speckle domains (Dellaire et al., 2002), using a sheep polyclonal antibody (PRP4K). Both anti-Flag antibodies (Flag, FIG. 3A) and the O-Sym aptamer (O-Sym, FIG. 3B) could specifically detect cells transfected with the LacI-SC35 fusion vector. The O-Sym aptamer could be used at concentrations between 50 to 100 nM for the detection of LacI-fusion proteins within transfected cells. The distribution of the LacI-SC35 fusion protein was indistinguishable from that of PRP4K, indicating that the fusion protein had been targeted correctly to nuclear speckle domains.

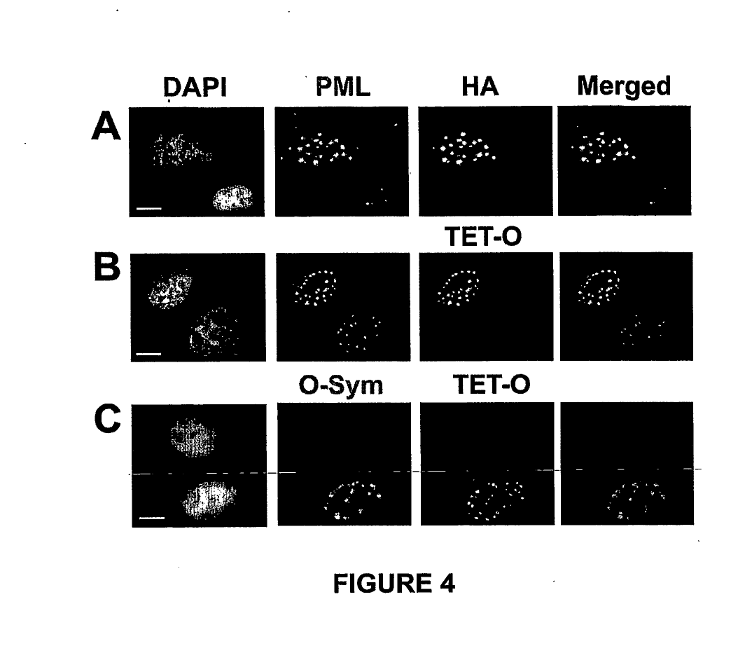

A second fusion protein vector containing the promyelocytic leukaemia protein (PML) fused with the Tet repressor (pGD-HA-TET-PML; FIG. 2D) was constructed. The PML protein is a structural component of PML nuclear bodies implicated in DNA repair, apoptosis, gene regulation, and tumour suppression (Salomoni and Pandolfi, 2002; Strudwick and Borden, 2002). PML nuclear bodies are functionally, spatially and biochemically distinct from nuclear speckles and thus a fusion protein targeted to this sub-nuclear compartment should not co-localise with a protein directed to nuclear speckles containing SC35. SK-N-SH cells were transfected with pGD-HA-TET-PML alone or in combination with pGD-Flag-Lac-SC35. After 24 hours post transfection, cells were fixed and analysed by immunofluorescence using antibodies directed against the HA epitope or with the Tet-O aptamer (FIG. 4). This localisation was compared to that of endogenous PML, using an anti-PML rabbit polyclonal antibody (PML, FIGS. 4A and 4B). Both anti-HA antibodies (HA, FIG. 3A) and the Tet-O aptamer (TET-O, FIG. 4B) could specifically detect cells transfected with the TetR-PML fusion vector. The Tet-O aptamer could be used at concentrations between 50 to 100 nM, thus equalling the sensitivity of O-Sym aptamer detection of LacI-fusion proteins within transfected cells. The TetR-PML fusion protein localised correctly to PML bodies as demonstrated by co-localisation with endogenous PML (FIGS. 4A and 4B). Un-ambiguous detection and localisation TetR-PML and LacI-SC35 was accomplished within the same cell by hybridisation with both aptamers (Tet-O and O-Sym, respectively; FIG. 4C). As expected PML nuclear bodies containing TetR-PML and nuclear speckles containing LacI-SC35 did not co-localise and these results demonstrate the ability of Aptamer ID™ to detect multiple proteins within the same cell without cross-hybridisation.

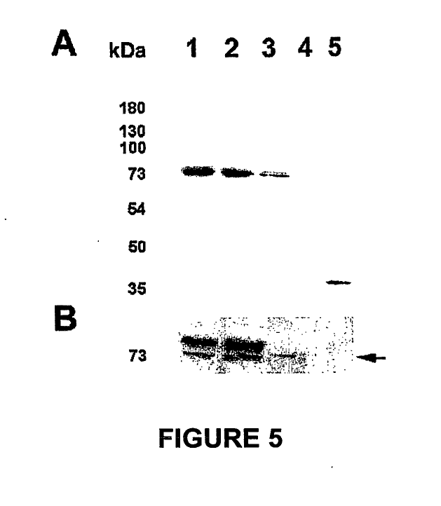

To demonstrate aptamer-protein isolation (Aptamer-PI; FIG. 1B) as a method of purifying recombinant proteins, whole cell lysates were made from SK-N-SH cells transiently expressing either LacI-tagged SC35 or LacI alone. These lysates were then used for Aptamer-PI using biotinylated O-Sym aptamers immobilised on streptavidin sepharose. The LacI protein also contains an N-terminal Flag-tag which was used for Western analysis of the Aptamer-PI purified LacI-SC35 (FIG. 5). Both LacI-SC35 and LacI alone were isolated by the O-Sym aptamer (Lanes 3 and 5 of FIG. 5A, respectively) but streptavidin sepharose alone (Lane 4, FIG. 5A) failed to isolate LacI-SC35. The identity of the LacI-SC35 protein was confirmed by Western analysis of the same blot using the anti-phospho-SR protein monoclonal antibody mAb 104 (FIG. 5B), which detects a number of SR proteins including SC35 (Roth et al., 1992, J. Cell Biol., 115, 587-596).

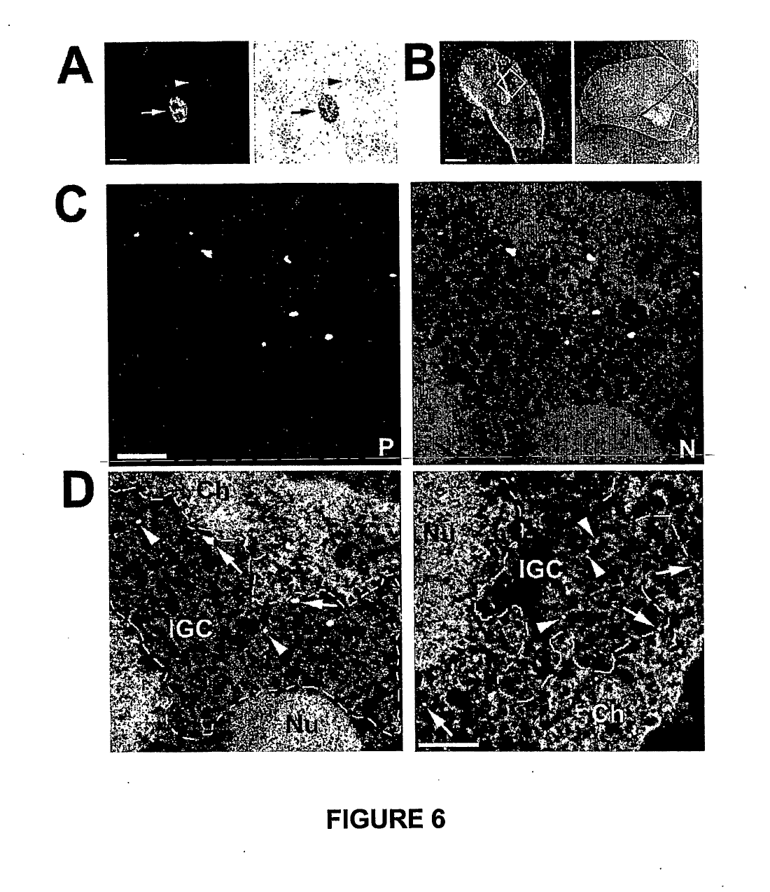

To demonstrate the utility of the Aptamer-ID detection system for correlative fluorescence and electron microscopy, a fluorogold aptamer was generated. This aptamer contains a Cy3 moiety on the 5′ end of one strand of the O-Sym oligonucleotide for detection in the fluorescence microscope, and an undecagold nanoparticle on the 5′ end of the complementary strand for detection by electron. microscopy. The nanogold label by itself is too small to be detected by conventional transmission electron microscopy, but can easily be enlarged for detection by silver enhancement. To illustrate the use of the doubly-labelled fluorogold apatamer for detection by correlative LM and EM, SK-N-SH neuroblastoma cells were transfected with pGD-Flag-Lac-SC35 and detected with the fluorogold aptamer as shown in FIG. 6A. The transfected cell (left panel) exhibited the distinct speckled pattern of the SC35 domain (Spector, D. L. Curr. Opin. Cell Biol., 5, 442-447), illustrating the effectiveness of the fluorescent component of the aptamer. The presence of gold in the aptamer was confirmed by performing an extended silver enhancement on the sample and bright field imaging by the light microscope (FIG. 6A (right panel)). Whereas transfected cells are heavily labelled with silver, untransfected cells have only background levels of silver deposition.

Correlative fluorescence and electron spectroscopic imaging (ESI)(5) was then carried out on LacI-SC35 transfected cells (see Materials and Methods). Physical sections were first imaged with the fluorescence microscope to detect the Cy3 label bound to the aptamer. The section containing the same cell was then imaged in the electron microscope. Electron spectroscopic imaging was choosen rather than conventional transmission electron microscopy because of its advantages for identifying protein- vs. nucleic acid-based components in and around nuclear speckle domains (also known as interchromatin granule clusters or IGCs). An overlay of fluorescence microscope images and low magnification energy filtered images were obtained (FIG. 6B). Only cells that expressed the LacI-SC35 fusion protein had detectable silver-enhanced fluorogold aptamer and this signal was restricted to regions of Cy3 signal in the correlative fluorescence image (FIG. 6B). A phosphorus map (FIG. 6C (left)) and a nitrogen map (FIG. 6C (right)) can be used to identify condensed chromatin, the nucleolus and the IGC domain. The fluorogold aptamer was heavily enhanced with silver, resulting in large accumulation of silver, shown as white blobs (FIG. 6C). Examination of the low magnification image also revealed the large silver clusters (FIG. 6B (left panel)). The composite of the phosphorus and nitrogen images (FIG. 6D (left panel) shows chromatin (yellow regions) and a morphologically dense region, corresponding to the nucleolus (yellow-green). The green structures, including the fibrous material in the IGC signifies protein-based structures. The area delineated by a dashed line corresponds to the region occupied by the IGC. The gold particles within the interior of this domain are highlighted by arrowheads, whereas the particles having close proximity to neighbouring chromatin are highlighted with arrows.

In a second nucleus presented, the gold particles were enhanced to a lesser degree (FIG. 6B (right panel)), and imaged by phosphorus and nitrogen mapping at higher magnification (FIG. 6D (composite shown in right panel)). The silver particles are much smaller but more uniform, thus more suitable for ultrastructure characterisation of protein components. Aptamer revealing the presence of SC35 in the core of the IGC are indicated with arrowheads, whereas aptamer revealing the presence of SC35 at the IGC:chromatin boundary are indicated with arrows.

The method of the invention can be provided as a kit where a specific vector is provided with a construct therein comprising one or more peptide tag and selected cDNA sequence encoding a selected protein. The kit is further provided with the aptamer that is directed to the binding of the peptide tag within the vector. Such kit may be suitably packaged and provided with instructions for use. Such a kit may also contain more than one vector.

The method of the invention can further be modified to provide the aptamer bound or coated onto a nitrocellulose membrane for large scale fusion protein expression screening. Suitable nitrocellulose membranes are well known to those of skill in the art.

The methodology of the present invention may be incorporated into DNA microarray (for example a cDNA array by BD Biosciences™), oligonucleotide arrays (for example, GeneChip™ by Affymetrix) or DNA chip technology that is typically fabricated on glass or nylon substrates, for which the aptamers are used to determine complementary binding to the peptide tag fusion. Such arrays are known to those of skill in the art. This may allow researchers information on several proteins and protein complexes simultaneously. The DNA microarray technology encompasses two formats that may be incorporated with the present invention. One is where the arrayed DNA sequence (i.e. the aptamer) is known and is immobilized to a solid surface such as glass using robot spotting and exposed to a set of targets (i.e. contain the fusion protein containing the peptide tag) either separately or in a mixture. This is the traditional DNA microarray method (R. Ekins and F. W. Chu, Microarrays: their origins and applications. Trends in Biotechnology, 1999, 17, 217-218). In another format, an array of the aptamer may be provided synthesized in situ (on-chip) or by conventional synthesis followed by on-chip immobilization and labelled. This array is exposed to a sample containing the fusion, hybridized, and the identity/abundance/location of complementary sequences are determined.

Furthermore, proteins having enzymatic activity such as peroxidases, kinases, phosphatases, acetyltransferases, methyltransferases etc., can be immobilized on chips or other solid supports (i.e. microtiter plates) using the method of the invention for ELISA assays or other immunoabsorbance assays. In addition, substrates for peptides or proteins immobilized on such chips or solid support using the method of the invention can be used to find novel substrates for peroxidases, kinases, phosphatases, acetyltransferases, methyltransferases and the like.

Protein-protein interactions can also be analyzed for peptides immobilized on beads, chips or other solid supports using the method of the invention which can then be combined with known methods of proteomic analysis, two-dimensional gel electrophoresis, western blotting and mass spectrometry.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Vector Construction and Aptamer Preparation

The mammalian expression vector pGD-Flag-Lac338 and its derivative pGD-Flag-Lac-SC35 were constructed as follows (FIGS. 1A and B). Plasmid pcDNA3.1/His-C (Invitrogen) was cut with HindIII and Asp718 and ligated to an Asp718/HindIII cut PCR product (Flag-Lac338) to produce pGD-Flag-Lac338. The Flag-Lac338 PCR product encodes the amino acid sequence of the Flag epitope (MDYKDDDDK) fused to the first 338 amino acids of the Lac repressor (LacI) and was amplified from the vector p3′SS-GFP-Lac-NLS (Robinett et al., 1996) using the following primers:

(Sequence ID No. 1)

LACFLAG-1 (tgacgtaagcttaggatggactataaagacgatgacgat
aaaccagtaacgttatacga);
and

(Sequence ID No. 2)

LAC3R-338 (ctataaggtaccgccccctccacttccaccgcccccaga
ggcggtttgcgtattgggcgcca).

To generate pGD-Flag-Lac-SC35, both pGD-Flag-Lac338 and the vector pBSK-SC35 were first cut with Asp718, blunt ended with Klenow in the same buffer, followed by phenol/chlorophorm extraction and precipitation, after which the DNA was resuspended and digested with BamHI. The resulting blunt end/BamHI pGD-Flag-Lac338 vector and the fragment containing the SC35 cDNA from pBSK-SC35 were ligated together. pBSK-SC35 was generated by subcloning of the human SC35 HindIII fragment from pEGFP-SC35 (gift of M. Hendzel) into HindIII cut pBlueScript(+) (Stratagene).

The mammalian expression vector pGD-HA-TET and its derivative pGD-HA-TET-PML were constructed as follows (FIGS. 1C and D). Plasmid pcDNA3.1/His-C (Invitrogen) was cut with HindIII and Asp718 and ligated to an Asp718/HindIII cut PCR product (HA-TET) to produce pGD-HA-TET. The HA-TET PCR product encodes the amino acid sequence of the hemagglutanin (HA) epitope (MGYPYDVPDYAG)(Sequence ID No. 3) fused to the TET repressor (TetR) and was amplified from the vector pTET-OFF (Clontech) using the following primers, TET-HA-1F (gggttttaagcttaccatgggatatccctatgatgtgccagactacgcgggaatgtctagattagataaaagt) (Sequence ID No. 4) and TET-HA-1R (tagattggatccaccgcctcctttaagttgtttttctaatccgca) (Sequence ID No. 5). To generate pGD-HA-TET-PML, both pGD-HA-TET and the vector pBSK-PML IV were first cut with BamHI and EcoRI, after which the linearized DNA fragment containing the human PML IV gene was gel purified and ligated to the linearized pGD-HA-TET. pBSK-PML IV was generated by subcloning the BamHI/EcoRI DNA fragment of the human PML IV gene from pDsRED-PML into BamHI/EcoRI digested pBlueScript(+) (Stratagene).

For construction of functionalised aptamers recognised by LacI, two 41 bp single stranded oligonucleotides were constructed encoding the symmetrical Lac operator 19 bp core sequence O-Sym (Lewis et al., 2002): O-Sym-1 (*gcgtgtgccagaattgtgagcgctcacaatttcttgaatct) (Sequence ID No. 6) and O-Sym-2 (*agattcaagaaattgtgagcgctcacaattctggctcacgc) (Sequence ID No.7); where * represents 5′ modification with either biotin or Cy3 (Sigma). O-Sym-1 and 2 were resuspended to 200 μM and equal volumes of each oligo were added to ⅕^{th} volume of 10× annealing buffer (50 mM Tris-Hcl pH 7.5, 1M NaCl, 0.2 mM EDTA). The oligo mixture was then boiled for 4 min at 90° C. followed by slow equilibration to room temperature to allow the annealing of the two O-Sym oligos to produce a ˜91 uM solution of the double stranded O-Sym aptamer.

Similarly, functionalised aptamers recognised by TetR, were constructed from the following 41 bp oligonucleotides: Tet-O-1 (*tcgagtttactccctatcagtgatagagaacgtatgtcgcc) (Sequence ID No. 8) and Tet-O-2 (*ggcgacatacgttctctatcactgatagggagtaaactcgt) (Sequence ID No. 9); where * represents 5′ modification with either biotin or Cy5 (Sigma). These oligonucleotides where annealed as above to produce a ˜91 μM solution of double stranded Tet-O aptamer.

### Example 2

### Cell Culture and Transfection

SK-N-SH cells were cultured according to the American Type Culture Collection (ATCC) guidelines for each cell line. Cells were split the day before transfection and 2×10⁵ cells were seeded at 10⁵ cells/ml onto 18 mm square coverslips in 8 or 6 well plates. The following day cells were transfected with 1-2 μg of pGD-Flag-Lac338 and pGD-TET-PML DNA alone or combined per well using Lipofectamine 2000 (Invitrogen™) as suggested by the manufacturer.

### Example 3

### Aptamer Hybridisation, Immunofluorescence and Microscopic Imaging of LacI and TetR-Tagged Proteins

Twenty-four to 36 hours post transfection cells were fixed in 1% paraformaldehyde in PBS for 5 min. followed by permeabilisation in 0.5% Triton X-100 for 5 min. at RT. Following several washes in PBS, cells were then blocked for 20 min. at RT with O-Sym Binding/Blocking (OSB) buffer (10 mM Tris-HCl pH 7.5, 0.1 mM EDTA, 150 mM KCl, 600 μg/ml sheared Herring sperm DNA, 200 μg/ml BSA). Directly following the blocking step, cells were hybridised for 1-2 hours at 37° C. with the O-Sym aptamer (labelled with either Cy3, biotin or both) alone or combined with Tet-O aptamer (labelled with either Cy5, biotin or both) in OSB buffer at a concentration of 50-100 nM. After hybridisation with the O-Sym or Tet-O aptamer, coverslips were either washed with 3×PBS and mounted in anti-fade reagent for immediate immunofluorescence detection or were further processed for immunofluorescent localisation of PML, SC35 or PRP4 kinase (PRP4K) as previously described (Dellaire et al., 2002). Primary antibodies directed against the Flag epitope (1:250, mouse mAb M2, Sigma), HA epitope (1:1000, mouse mAb HA.11, Zymed), PML (1:200, rabbit anti-PML antibody, Chemicon) or PRP4K (1:100, sheep antibody H143, Dellaire et al., 2002) were subsequently detected with secondary antibodies conjugated to Cy5 (1:100, donkey anti-sheep and anti-mouse Abs) or to Cy3 (1:500, donkey anti-rabbit Ab)(Jackson Immuno Laboratories). Alternately, following hybridisation with a biotinylated O-Sym aptamer, the localisation of the LacI- or TetR-fusion protein was visualised using Cy3-Streptavidin (1:200, Sigma).

### Example 4

### Fluorogold Aptamer Synthesis

The aptamer consisting of the disulfide-modified O-Sym-1 and Cy3-modified O-Sym-2 was treated with 0.04 M DTT (0.17M Na₂HPO₄, pH 8.0) for 16 hours at room temperature to cleave the disulfide bond. The thiol by-products and DTT were removed using a Sephadex column (NAP-5, Amersham Biosciences) equilibrated with 20 mM Na₂HPO₄,150 NaCl, 1 mM EDTA, pH 6.5 (conjugation buffer). The aptamer was further purified by 70% ethanol precipitation and repeated washes. The pellet was resuspended in the conjugation buffer to a concentration of 100 μM. Then, 10 μl (1 nmol) of this solution was added to 10 nmol of monomaledo-undecagold reagent (Nanoprobes, Yaphank, N.Y.) in 1 ml of the conjugation buffer. The mixture was incubated at room temperature with stirring for 1 hour, then incubated at 4° C. for 16 hours. The functionalised aptamers were isolated from excess nanocrystals by ethanol precipitation with excess salmon sperm DNA, followed by repeated washes. The product was resuspended in 10 mM Tris-HCl, 0.1 mM EDTA, 150 mM KCl, pH 7.5, to achieve a final aptamer concentration of approximately 10 μM.

### Example 5

### Protein Isolation by Aptamer-PI and Western Analysis

SK-N-SH neuroblastoma cells transfected with either LacI or LacI-SC35 were lysed by sonication (3×30 s at 20% power using an Ultrasonic Processor (Hert Systems)) in Aptamer-PI buffer (A-PIB; 20 mM Hepes pH 7.5, 250 mM KCl, 10% glycerol, 1 mM phenyl-methylsulfonyl fluoride (PMSF), 1× Complete protease cocktail (Roche), 1 mM NaF, 40 mM β-glycerolphophate). The resulting lysate (PI lysate) was then centrifuged at 12,000 G for 20 min to remove cellular debris and pre-cleared by incubation of the supernatant with streptavidin sepharose beads (Invitrogen) for 1 h at 4° C. followed by centrifugation at 12,000 G for 5 s to remove the sepharose beads. The precleared PI lysate was either snap frozen on dry ice or used immediately for Aptamer-PI. For Aptamer-PI, streptavidin sepharose was first pre-incubated with biotinylated O-Sym aptamers (100 μl of streptavidin sepharose in 1 ml of PBS containing 500 nM aptamer) and then washed 3× with A-PIB. Then PI lysate containing 250-500 μg of total protein was incubated with 30-50 μl of streptavidin sepharose beads pre-incubated with biotinylated O-Sym aptamer over night at 4° C. Mock protein isolation (PI) was carried out using streptavidin sepharose without aptamer. Aptamer-PIs were then washed 3× with A-PIB containing 0.5% Triton-X 100 and 2× with PBS before SDS-PAGE and Western transfer. Western blots were visualised using the SuperSignal West Pico Chemiluminescent kit (Pierce) as per the manufacturer's instructions.

Alternatively, qualitative analysis of the level of purification of LacI-SC35 by Aptamer-PI, versus immunoprecipitation using an antibody, was carried out by SDS-PAGE, followed by Coomassie Blue staining of the acrylamide gel to visualise the isolated proteins. Briefly, 500-1000 μg of total protein lysate prepared from LacI-SC35 transfected SK-N-SH, as described above, was first precleared twice by incubation with 2×100 μl of streptavidin sepharose 1 h at 4° C. The precleared lysate was then incubated overnight at 4° C. with continuous mixing with either 100 μl of streptavidin sepharose alone (Mock) or pre-incubated with O-Sym aptamer (Aptamer-PI) or 50 μl of Protein G sepharose pre-incubated with 20 μg of anti-Flag epitope antibody M2 (Sigma)(immunoprecipitation or IP). In addition, unrelated dsDNA aptamer (i.e. TetO) was added to a concentration of ˜1 μM to the precleared lysate during Aptamer-PI to reduce the non-specific co-purification of other DNA binding proteins. The Aptamer-PI and anti-Flag IP were then washed 3× with A-PIB containing 0.5% Triton-X 100 and 2× with PBS before being boiled for SDS-PAGE followed by staining of the gel by Coomassie Blue.

### Example 6

### Correlative Light and Electron Spectroscopic Imaging (LM/ESI) of Fluorogold Aptamer-ID

SK-N-SH cells transfected with LacI-SC35 were labelled by Aptamer-ID as above using fluorogold O-Sym aptamers. After labelling, cells were post-fixed (8% paraformaldehyde, 2% glutaraldehyde for 5 min at RT) and subjected to silver enhancement of the fluorogold aptamers for 30 min at RT using a either a silver enhancement kit for LM or EM (Electron Microscopy Sciences). The EM enhancement was performed 3 times, using fresh enhancement solution each time. The cells were then dehydrated in a series of graded ethanol washes (30%, 50%, 70% and 95%) before Quetol 651 resin (EM Science) infiltration, curing and sectioning, as previously described (Dellaire, et al., 2004, Methods Enzymol. 375, 456-478; Ren et al., 2003, J Histochem Cytochem., 51, 605-612). Images of cells of interest were collected by both LM, as described above, and electron spectroscopic imaging, using a Tecnal 20 TEM (FEI) at 200 kV, equipped with an imaging spectrometer (Gatan). Elemental maps were generated by dividing the element-enhanced post-edge image by the pre-edge image following alignment by cross-correlation. Net ratio elemental maps were derived from pre- and post-edge images recorded at 120 and 155 eV (L_{II,III} edge) for phosphorus, and at 385 and 415 eV (K edge) for nitrogen. The recording times required to obtain the pre-edge and post-edge images are in the range of 10 to 30 seconds. The images were processed using Digital Micrograph (Gatan) and Photoshop 6.0/7.0 (Adobe).

### REFERENCES

Bacher, J. M. and A. D. Ellington. (1998) Drugs Discovery Today 3:265-273

Bianchini, M., M. Radrizzani, M. G. Brocardo, G. B. Reyes, C. G. Solveyra, T. A. Santa-Coloma. (2001) J. Immunol. Methods 252:191-197

Blank, M., T. Weinshenk, M. Priemer and H. Schluesener. (2001) J. Biol. Chem. 276:16464-16468

Bock, L. C., L. C. Griffin, J. A. Latham, E. H. Vermaas, and J. J. Toole. (1992) Nature 355:564-566.

Brody, E. N. and L. Gold. (2000) J. Biotechnol. 74:5-13

Dellaire, G., E. M. Makarov, J. J. Cowger, D. Longman, H. G. E. Sutherland, R. Luhrmann, J. Torchia and W. A. Bickmore. (2002) Mol. Cell. Biol. 22:5141-5156

Dougan, H., J. I. Weitz, A. R. Stafford, K. D. Gillespie, P. Klement, J. B. Hobbs, D. M. Lyster. (2003) Nucl. Med. Biol. 30:61-72.

Ellington, A. D and J. W. Szostak. (1990) Nature 346:818-22.

Grkovic, S., M. H. Brown and R. A. Skurray (2002) Microbiol. Mol. Biol. Rev. 66:671-701

Hermann, T. and D. J. Patel. (2000) Science 287:820-825

Hillen, W., and C. Berens. (1994) Annu Rev. Microbiol. 48:345-369

Hsieh, M. and M. Brenowitz (1997) J. Biol. Chem. 272:22092-22096

Jayasena, S. D. (1999) Clin. Chem. 45:1628-1650

Kercher, M. A., P. Lu, and M. Lewis. (1997) Curr. Opin. Struct. Biol. 7:76-85

Lewis, M., G. Chang, N. C. Horton, M. A. Kercher, H. C. Pace, M. Z. Schumacher, R. G. Brennan, and P. Lu. (1996) Science 271:1247-1254

Michaelis, C., Ciosk, R., and Nasmyth, K. (1997) Cell 91(1):35-45.

Morris, K. N., K. B. Jensen, C. M. Julin, M. Well, and L. Gold. (1998) Proc. Natl. Acad. Sci. USA 95:2902-2907

Nguyen, C. C., and M. H. Saier, Jr. (1995) Febs Lett. 377:98-102

Pines, 3. (1995) Trends Genet. 11:326-327

Robinett, C. C., A. Straight, G. Li, C. Willhelm, G. Sudlow, A. Murray, and A. S. Belmont. (1996) J. Cell Biol. 135:1685-1700

Roulet, E., S. Busso, A. A. Camargo, A. J. Simpson, N. Mermod, and P. Bucher. (2002) Nat. Biotechnol. 20:831-835

Salomoni, P., and P. P. Pandolfi. (2002) Cell 108:165-170

Schrock, E., du Manoir, S., Veldman, T., Schoell, B., Wienberg, J., Ferguson-Smith, M. A., Ning, Y., Ledbetter, D. H., Bar-Am, I., Soenksen, D., Garini, Y., and Ried, T. (1996) Science 273(5274):494-497.

Spector, D. L. (1993) Curr. Opin. Cell Biol. 5:442-447

Speicher, M. R., Gwyn Ballard, S., and Ward, D. C. (1996) Nat. Genet. 12(4): 368-375.

Stanlis, K. K. H. and J. R. McIntosh. (2003) J. Histo. Cyto. 51:797-808

Strudwick, S., and K. L. Borden. (2002) Leukaemia 16:1906-1917

Terpe, K. (2003) Appl. Microbiol. Biotechnol. 60:523-533

Tuerk, C and L. Gold. (1990) Science 249:505-510

van Roessel, P., and A. H. Brand. (2002) Nat. Cell Biol. 4:E15-E20

Although preferred embodiments have been described herein in detail it is understood by those of skill in the art that using no more than routine experimentation, many equivalents to the specific embodiments of the invention described herein can be made. Such equivalents are intended to be encompassed by the scope of the claims appended hereto.

1. A method for detecting a protein in situ, the method comprising contacting a cell expressed peptide tag fusion protein with a nucleic acid aptamer that recognizes the peptide tag with high affinity and forms a complex, and detecting the complex.

2. The method of claim 1, wherein said aptamer is selected from the group consisting of a single stranded DNA, double stranded DNA, hairpin DNA, single stranded RNA, double stranded RNA, hairpin RNA, protein nucleic acid aptamer or a hybrid combination thereof.

3. The method of claim 2, wherein said nucleic acid aptamer is double stranded DNA.

4. The method of claim 2, wherein said aptamer is chemically modified.

5. The method of claim 4, wherein said chemical modification is selected from the group consisting of reactive thiols, amines, cobalt or iron paramagenetic beads, fluorophores, quantum dots, peptides, metal chelating peptides or compounds, gold particles, simple or complex sugars, biotin and combinations thereof.

6. The method of claim 1, wherein said peptide tag is selected from the group consisting of a natural or engineered sequence of a virus, archaebacteria, eukaryotic and a prokaryotic organism.

7. The method of claim 6, wherein said peptide tag is a prokaryotic DNA binding protein.

8. The method of claim 7, wherein said prokaryotic DNA binding protein is selected from the group consisting of Lac repressor, Tet repressor, fructose repressor, purine repressor,galactose repressor, AraC repressor, MerR repressor, MarR repressor, BmrR repressor, QacR repressor and EmrR repressor.

9. The method of claim 6, wherein said peptide tag is modified by a modification selected from the group consisting of methylation, acetylation, phosphorylation, ADP-ribosylation, sumolation, ubiquitination, glycosylation and any combination thereof.

10. The method of claim 8, wherein said DNA binding protein is a Lac repressor.

11. The method of claim 1, wherein said cell is selected from the group consisting of archebacterial cell, prokaryotic cell and eukaryotic cell.

12. The method of claim 11, wherein said cell is a eukaryotic cell.

13. The method of claim 12, wherein said cell is a mammalian cell.

14. The method of claim 1, wherein prior to contacting, said cell is fixed and permeabilized.

15. The method of claim 5, wherein detecting is done by immunofluorescence.

16. The method of claim 1, wherein detection of said complex is visualized in a sub-cellular and/or sub-nuclear location within said cell.

17. The method of claim 1, wherein said aptamer is provided bound to a nitrocellulose membrane.

18. The method of claim 1, wherein said aptamer is provided as a microarray on a substrate.

19. A method for detecting proteins in situ in a cell, the method comprising;
a) preparing a protein/peptide tag fusion vector;
b) transforming a cell with a);
c) contacting b) with a chemically modified nucleic acid aptamer, wherein said protein tag is recognized by said nucleic acid aptamer and forms a complex; and
d) detecting said complex.

20. The method of claim 19, wherein said aptamer is selected from the group consisting of a single stranded DNA, double stranded DNA, hairpin DNA, single stranded RNA, double stranded RNA, hairpin RNA, protein nucleic acid aptamer or a hybrid combination thereof.

21. The method of claim 20, wherein said nucleic acid aptamer is double stranded DNA.

22. The method of claim 19, wherein said chemical modification is selected from the group consisting of reactive thiols, amines, cobalt or iron paramagenetic beads, fluorophores, quantum dots, peptides, gold particles, metal chelating peptides or compounds, simple or complex sugars, biotin and combinations thereof.

23. The method of claim 19, wherein said peptide tag is selected from the group consisting of a natural or engineered sequence of a virus, archaebacteria, eukaryotic organism and a prokaryotic organism.

24. The method of claim 23, wherein said peptide tag is a prokaryotic DNA binding protein.

25. The method of claim 22, wherein said prokaryotic DNA binding protein is selected from the group consisting of the Lac repressor, Tet repressor, fructose repressor, purine repressor, galactose repressor, AraC repressor, MerR repressor, MarR repressor, BmrR repressor, QacR repressor and EmrR repressor.

26. The method of claim 19, wherein said peptide tag is modified by a modification selected from the group consisting of methylation, acetylation, phosphorylation, ADP-ribosylation, sumolation, ubiquitination, glycosylation, hydroxylation and any combination thereof.

27. The method of claim 25, wherein said DNA binding protein is a Lac repressor.

28. The method of claim 19, wherein said cell is selected from the group consisting of archebacterial cell, prokaryotic cell and eukaryotic cell.

29. The method of claim 28, wherein said cell is a eukaryotic cell.

30. The method of claim 29, wherein said cell is a mammalian cell.

31. The method of claim 19, wherein prior to step c), b) is fixed and permeabilized.

32. The method of claim 19, wherein said complex is detected in a sub-cellular and/or sub-nuclear structure.

33. The method of claim 19, wherein detection is done by immunofluorescence.

34. A method for the detection of one or more proteins and/or protein complexes in situ, the method comprising:
contacting a transfected cell expressing a fusion protein comprising a prokaryotic DNA binding protein with a functionalized nucleic acid aptamer, wherein said prokaryotic DNA binding protein is recognized by said functionalized nucleic acid aptamer to form a complex, and detecting said complex.

35. The method of claim 34, wherein said nucleic acid aptamer is selected from the group consisting of a single stranded DNA, double stranded DNA, hairpin DNA, single stranded RNA, double stranded RNA, hairpin RNA, protein nucleic acid aptamer or a hybrid combination thereof.

36. The method of claim 35, wherein said nucleic acid aptamer is double stranded DNA.

37. The method of claim 35, wherein said functionalized nucleic acid aptamer is functionalized with a group selected from the group consisting of reactive thiols, amines, cobalt or iron paramagnetic beads, fluorophores, quantum dots, peptides, metal chelating peptides or compounds, simple or complex sugars, biotin and combinations thereof.

38. The method of claim 35, wherein said cell is selected from the group consisting of archebacterial cell, prokaryotic cell and eukaryotic cell.

39. The method of claim 38, wherein said cell is a eukaryotic cell.

40. The method of claim 39, wherein said cell is a mammalian cell.

41. A method for the detection and purification of one or more proteins and/or protein complexes, the method comprising:
a) transforming a cell with a protein/peptide tag fusion vector;
b) culturing a) to express a fusion protein comprising a desired protein fused to a protein tag;
c) making a cellular extract from b);
d) contacting c) with a nucleic acid aptamer bound to a solid matrix or beads, wherein said protein tag is recognized by said nucleic acid aptamer to form a complex; and
e) purifying said complex from d).

42. The method of claim 41, wherein said nucleic acid aptamer is selected from a single stranded or double stranded DNA, RNA, protein nucleic acid aptamer or a hybrid combination thereof.

43. The method of claim 42, wherein said nucleic acid aptamer is a double stranded DNA aptamer wholey or in part.

44. The method of claim 35, wherein said nucleic acid aptamer is functionalized with a group selected from the group consisting of reactive thiols, amines, cobalt or iron paramagnetic beads, fluorophores, quantum dots, peptides, metal chelating peptides or compounds, simple or complex sugars, biotin and combinations thereof.

45. The method of claim 41, wherein said cell is selected from the group consisting of archebacterial cell, prokaryotic cell and eukaryotic cell.

46. The method of claim 45, wherein said cell is a eukaryotic cell.

47. The method of claim 46, wherein said cell is a mammalian cell.

48. The method of claim 41, wherein said peptide tag is selected from the group consisting of a natural or engineered sequence of a virus, archaebacteria, eukaryotic organism and a prokaryotic organism.

49. The method of claim 48, wherein said peptide tag is a prokaryotic DNA binding protein.

50. The method of claim 49, wherein said prokaryotic DNA binding protein is selected from the group consisting of the Lac repressor, Tet repressor, fructose repressor, purine repressor and galactose repressor.

51. The method of claim 49, wherein said peptide tag is modified by a modification selected from the group consisting of methylation, acetylation, phosphorylation, ADP-ribosylation, sumolation, ubiquitination, glycosylation, hydoxylation and any combination thereof.

52. The method of claim 41, wherein said aptamer is covalently bound to sepharose beads.

53. The method of claim 41, wherein said aptamer is coupled to a paramagnetic particle.

54. (canceled)


SEQUENCE LISTING (9 sequences)

SEQ ID NO: 1 - DNA, 59 bp, Artificial Sequence
  Primer
tgacgtaagcttaggatggactataaagacgatgacgataaaccagtaacgttatacga

SEQ ID NO: 2 - DNA, 62 bp, Artificial Sequence
  Primer
ctataaggtaccgccccctccacttccaccgcccccagaggcggtttgcgtattgggcgcca

SEQ ID NO: 3 - protein, 12 aa, Artificial Sequence
  Chemically Synthesized
MGYPYDVPDYAG

SEQ ID NO: 4 - DNA, 73 bp, Artificial Sequence
  Primer
gggttttaagcttaccatgggatatccctatgatgtgccagactacgcgggaatgtctagattagataaaagt

SEQ ID NO: 5 - DNA, 45 bp, Artificial Sequence
  Primer
tagattggatccaccgcctcctttaagttgtttttctaatccgca

SEQ ID NO: 6 - DNA, 41 bp, Artificial Sequence
  Primer
gcgtgtgccagaattgtgagcgctcacaatttcttgaatct

SEQ ID NO: 7 - DNA, 41 bp, Artificial Sequence
  Primer
agattcaagaaattgtgagcgctcacaattctggctcacgc

SEQ ID NO: 8 - DNA, 41 bp, Artificial Sequence
  Primer
tcgagtttactccctatcagtgatagagaacgtatgtcgcc

SEQ ID NO: 9 - DNA, 41 bp, Artificial Sequence
  Primer
ggcgacatacgttctctatcactgatagggagtaaactcgt